(19) United States
(12) Patent Application Publication
(10) Pub. No.: US 2023/0142121 A1
(43) Pub. Date: May 11, 2023

(54) FUSION OF SENSOR DATA FOR PERSISTENT DISEASE MONITORING

(71) Applicant: CHEMIMAGE CORPORATION, Pittsburgh, PA (US)

(72) Inventors: Patrick J. TREADO, Pittsburgh, PA (US); Aaron SMITH, Monroeville, PA (US); Heather E. GOMER, Sewickley, PA (US); Lewis L. LANKER, Pittsburgh, PA (US); Jeffrey K. COHEN, Pittsburgh, PA (US); Matthew P. NELSON, Harrison City, PA (US); Margo MALONE, Pittsburgh, PA (US); Elizabeth EZAR, Pittsburgh, PA (US)

(21) Appl. No.: 18/052,185

(22) Filed: Nov. 2, 2022

Related U.S. Application Data
(60) Provisional application No. 63/274,783, filed on Nov. 2, 2021.
(60) Provisional application No. 63/284,985, filed on Dec. 1, 2021.

(51) Int. Cl. G16H 50/20; G06T 7/00; G06V 10/141; G06V 10/60; A61B 5/1455; A61B 5/318; G06F 18/25

(52) U.S. Cl. CPC G16H 50/20; G06T 7/0012; G06V 10/141; G06V 10/60; A61B 5/14551; A61B 5/318; G06F 18/251

(57) ABSTRACT

A method for improved disease monitoring is disclosed. The method includes receiving image data from an image sensor configured for monitoring a hypothesis disease in a patient. Additional data is received from one or more additional sensors configured to monitor one or more factors related to disease activity of the patient. The received image data is fused with the received additional data from the one or more additional sensors to generate fused data set. A disease condition for the patient is determined based on the fused data set. A disease monitoring computing device and non-transitory medium are also disclosed.

### CROSS-REFERENCE TO RELATED APPLICATIONS

This application claims priority to U.S. Provisional Application Ser. No. 63/274,783 filed on Nov. 2, 2021 and U.S. Provisional Application Ser. No. 63/284,985 filed on Dec. 1, 2021, the entireties of which are hereby incorporated by reference in their entireties.

### TECHNICAL FIELD

The present disclosure relates generally to systems and methods for improved disease monitoring.

### BACKGROUND

There are a variety of diseases that are difficult to diagnose. The difficulty arises from a variety of factors, but in general such diseases frequently do not present symptoms during their early stages and slowly become more debilitating over time. Conventionally, these diseases have been monitored by a combination of medical history, patient interviews, and periodic testing that can be slow, invasive, or otherwise inconvenient. There is a need for systems that can provide early, objective, accurate, and frequent monitoring for advance warning of potential disease.

### SUMMARY

Embodiments related to the long-term, persistent monitoring and identification of potential disease (that is, a hypothesis disease) are disclosed.

The disclosure of U.S. patent application Ser. No. 17/335,857 filed Jun. 1, 2021 and entitled “A METHOD FOR FUSING SENSOR DATA FOR CARDIAC MONITORING AND DEVICES THEREOF” is hereby incorporated by reference in its entirety.

In some aspects, the techniques described herein relate to a method of disease monitoring, the method including: receiving, by a disease monitoring computing device, image data from an image sensor configured for monitoring a hypothesis disease in a patient; receiving, by the disease monitoring computing device, additional data from one or more additional sensors configured to monitor one or more factors related to disease activity of the patient; fusing, by the disease monitoring computing device, the received image data with the received additional data from the one or more additional sensors to generate a fused data set; and determining, by the disease monitoring computing device, a hypothesis disease for the patient based on the fused data set.

In some aspects, the techniques described herein relate to a method, wherein fusing the received image data with the received additional data further includes: applying, by the disease monitoring computing device, one or more data fusion algorithms to the received image data and the received additional data.

In some aspects, the techniques described herein relate to a method, wherein applying the data fusion algorithm includes utilizing one or more of an image weighted Bayesian function, logistic regression, linear regression, regression with regularization, naïve Bayes, classification and regression tress, support vector machines, or a neural network.

In some aspects, the techniques described herein relate to a method, wherein the received additional data is non-image data.

In some aspects, the techniques described herein relate to a method, wherein the one or more additional sensors include one or more of a pulse oximeter, an electrocardiogram machine, a sensor for thoracic impedance, or an implantable disease monitoring device.

In some aspects, the techniques described herein relate to a method, wherein determining the hypothesis disease further includes: identifying, by the disease monitoring computing device, one or more indications of a potential heart failure.

In some aspects, the techniques described herein relate to a method, wherein the image sensor includes: a light source configured to irradiate a tissue of the patient with light; and a detector configured to collect reflected light from the tissue of the patient and generate the image data associated with the reflected light; wherein the method further includes: receiving, by the disease monitoring computing device, the image data associated with the reflected light; calculating, by the disease monitoring computing device, intensity values for reflected light; and determining, by the disease monitoring computing device, whether the tissue exhibits symptoms of edema.

In some aspects, the techniques described herein relate to a method, wherein the tissue of the patient is located on a forearm of the patient.

In some aspects, the techniques described herein relate to a method, wherein the image data is spectral data and the image sensor is a spectral sensor.

In some aspects, the techniques described herein relate to a method, wherein the hypothesis disease includes at least one of lymphatic disease, kidney disease, peripheral vascular disease, protein deficiency (including protein S deficiency), chronic obstructive pulmonary disease, diabetes, sepsis, cancer such as breast cancer with lymph node metastasis, or stroke (including ischemic stroke, hemorrhagic stroke, or transient ischemic attack).

In some aspects, the techniques described herein relate to a non-transitory computer readable medium having stored thereon instructions for improved disease monitoring including executable code that, when executed by one or more processors, causes the one or more processors to: receive image data from an image sensor configured for monitoring edema in a patient; receive additional data from one or more additional sensors configured to monitor one or more factors related to disease activity of the patient; fuse the received image data with the received additional data from the one or more additional sensors to generate a fused data set; and determine a disease condition for the patient based on the fused data set.

In some aspects, the techniques described herein relate to a non-transitory computer readable medium, wherein the processors fuse the received image data with the received additional data by applying one or more data fusion algorithms to the received image data and the received additional data.

In some aspects, the techniques described herein relate to a non-transitory computer readable medium, wherein the data fusion algorithm includes one or more of an image weighted Bayesian function, logistic regression, linear regression, regression with regularization, naïve Bayes, classification and regression tress, support vector machines, or a neural network.

In some aspects, the techniques described herein relate to a non-transitory computer readable medium, wherein the additional data that the processors receive is non-image data.

In some aspects, the techniques described herein relate to a non-transitory computer readable medium, wherein the one or more additional sensors include one or more of a pulse oximeter, an electrocardiogram machine, a sensor for thoracic impedance, or an implantable disease monitoring device.

In some aspects, the techniques described herein relate to a non-transitory computer readable medium, wherein determination of the disease condition further includes: identifying one or more indications of lymphatic disease, kidney disease, peripheral vascular disease, protein deficiency (including protein S deficiency), chronic obstructive pulmonary disease, diabetes, sepsis, cancer such as breast cancer with lymph node metastasis, or stroke (including ischemic stroke, hemorrhagic stroke, or transient ischemic attack).

In some aspects, the techniques described herein relate to a disease monitoring computing device including memory including programmed instructions stored thereon for disease monitoring and one or more processors coupled to the memory and configured to execute the stored programmed instructions, which when the programmed instructions are executed the disease monitoring computing device: receives image data from an image sensor configured for monitoring edema in a patient; receives additional data from one or more additional sensors configured to monitor one or more factors related to disease activity of the patient; fuses the received image data with the received additional data from the one or more additional sensors to generate a fused data set; and determines a disease condition for the patient based on the fused data set.

In some aspects, the techniques described herein relate to a disease monitoring computing device, wherein the processors fuse the received image data with the received additional data by applying one or more data fusion algorithms to the received image data and the received additional data.

In some aspects, the techniques described herein relate to a disease monitoring computing device, wherein the data fusion algorithm includes one or more of an image weighted Bayesian function, logistic regression, linear regression, regression with regularization, naïve Bayes, classification and regression tress, support vector machines, or a neural network.

In some aspects, the techniques described herein relate to a disease monitoring computing device, wherein the additional data that the processors receive is non-image data.

In some aspects, the techniques described herein relate to a disease monitoring computing device, wherein the one or more additional sensors include one or more of a pulse oximeter, an electrocardiogram machine, a sensor for thoracic impedance, an implantable disease monitoring device, or a breathing rate device.

In some aspects, the techniques described herein relate to a disease monitoring computing device, wherein determining the disease condition further includes: identifying one or more indications including lymphatic disease, kidney disease, peripheral vascular disease, protein deficiency (including protein S deficiency), chronic obstructive pulmonary disease, diabetes, sepsis, cancer such as breast cancer with lymph node metastasis, or stroke (including ischemic stroke, hemorrhagic stroke, or transient ischemic attack).

In some aspects, the techniques described herein relate to a disease monitoring computing device, wherein the image sensor includes: a light source configured to irradiate a tissue of the patient with light; and a detector configured to collect reflected light from the tissue of the patient and generate the image data associated with the reflected light; wherein the disease monitoring computing device further: receives the image data associated with the reflected light; calculates intensity values for reflected light; and determines whether the tissue exhibits symptoms of edema.

In some aspects, the techniques described herein relate to a disease monitoring computing device, wherein the image data is spectral data and the image sensor is a spectral sensor.

### DRAWINGS

The accompanying drawings, which are incorporated in and form a part of the specification, illustrate the embodiments of the invention and together with the written description serve to explain the principles, characteristics, and features of the invention. In the drawings:

FIG. 1 a block diagram of an environment with an exemplary disease monitoring computing device;

FIG. 2 is a block diagram of the exemplary disease monitoring computing device of FIG. 1; and

FIG. 3 is a flowchart of an exemplary method for improved, disease monitoring using sensor data fusion.

### DETAILED DESCRIPTION

This disclosure is not limited to the particular systems, devices and methods described, as these may vary. The terminology used in the description is for the purpose of describing the particular versions or embodiments only, and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

The embodiments of the present teachings described below are not intended to be exhaustive or to limit the teachings to the precise forms disclosed in the following detailed description. Rather, the embodiments are chosen and described so that others skilled in the art may appreciate and understand the principles and practices of the present teachings.

As described above, there are a variety of diseases that can be monitored in accordance with the disclosure. As described herein, the diseases are referred to as hypothesis diseases. It should be noted that the hypothesis diseases are not limited and might not be present in a particular patient even though monitoring or testing is being performed on a continuous, randomly intermittent, or regularly intermittent basis. The hypothesis diseases include one or more of lymphatic disease, kidney disease, peripheral vascular disease, protein deficiency (including protein S deficiency), chronic obstructive pulmonary disease, diabetes, sepsis, cancer such as breast cancer with lymph node metastasis, or stroke (such as ischemic stroke, hemorrhagic stroke, or transient ischemic attack).

Alternatively, the disclosed systems, methods and apparatus has many applications for long term persistent monitoring, including at least one of atrial fibrillation, cardiac monitoring, sepsis, or monitoring hydration for general personal wellness or for ensuring athletic performance.

Disease Monitoring

As described above, the disclosure is directed to a variety of diseases which require persistent, sometimes continuous, monitoring. Atrial fibrillation is one of the most common types of arrhythmias, which are irregular heart rhythms. Atrial fibrillation causes a person's heart to beat significantly faster than is normal, and the chambers of the heart do not work together efficiently. During atrial fibrillation, a person will often feel tired or dizzy, or the person might notice heart palpitations or chest pain. Blood can also collect in the heart, which increases the risk of forming clots leading to strokes and other complications. Atrial fibrillation can last for short or long periods of time, and can arise from damage to heart tissue.

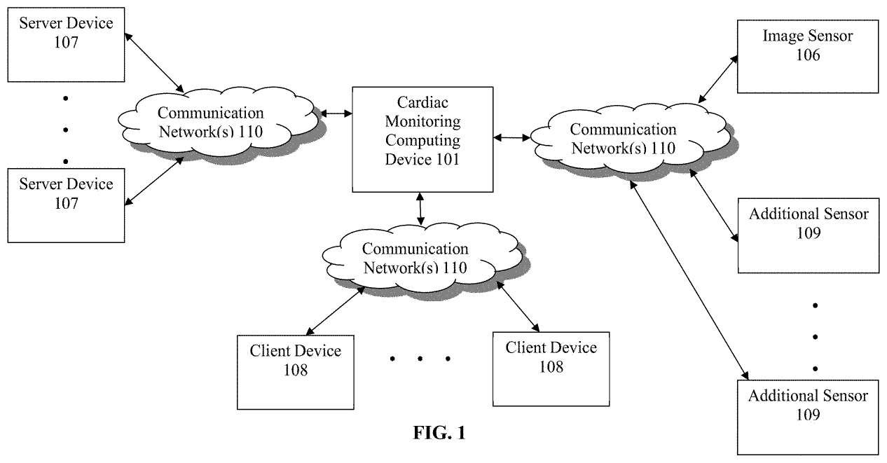

Sepsis is caused by many organisms including bacteria, viruses and fungi and is a life-threatening condition that arises when the body's response to infection causes injury to its own tissues and organs. Common signs and symptoms of sepsis include fever, increased heart rate, increased breathing rate, and confusion. Sepsis can be caused by the collection of fluid in a patient's abdomen, which can be a complication of surgery. Frequently, a drainage tube must be placed to drain excess fluid from the abdomen, and the amount of fluid in the abdomen must be closely monitored. Other symptoms of sepsis include urinating less than usual, nausea and vomiting, diarrhea, fatigue and weakness, blotchy or discolored skin, sweaty or clammy skin, and severe pain. Referring to FIG. 1, an exemplary environment with an exemplary disease monitoring computing device is illustrated. The disease monitoring computing device in this example is coupled to a plurality of server devices 107, a plurality of client devices 108, an image sensor 106, and a plurality of additional sensors 109 via communication network(s) 110, although the disease monitoring computing device 101, server devices 107, image sensor 106, and/or additional sensors 109 may be coupled together via other topologies. This technology provides a number of advantages including providing methods, non-transitory computer readable media, and disease monitoring computing device 101s that provide improved disease monitoring. In particular, certain implementations of this technology utilize data fusion from multiple sensors, including image data related to the hypothesis disease, to provide more accurate and reliable, early, and objective indications corresponding to the hypothesis disease.

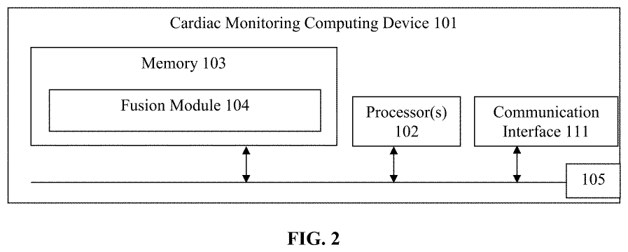

Referring to FIGS. 1-2, the disease monitoring computing device 101 in this example includes processor(s) 102, a memory 103, and/or a communication interface 111, which are coupled together by a bus or other communication link 105, although the disease monitoring computing device 101 can include other types and/or numbers of elements in other configurations. The processor(s) 102 of the disease monitoring computing device 101 may execute programmed instructions stored in the memory 103 for the any number of the functions described and illustrated herein. The processor(s) 102 of the disease monitoring computing device 101 may include one or more CPUs or general purpose processors with one or more processing cores, for example, although other types of processor(s) can also be used.

The memory 103 of the disease monitoring computing device 101 stores these programmed instructions for one or more aspects of the present technology as described and illustrated herein, although some or all of the programmed instructions could be stored elsewhere. A variety of different types of memory storage devices, such as random access memory (RAM), read only memory (ROM), hard disk, solid state drives, flash memory, or other computer readable medium which is read from and written to by a magnetic, optical, or other reading and writing system that is coupled to the processor(s), can be used for the memory.

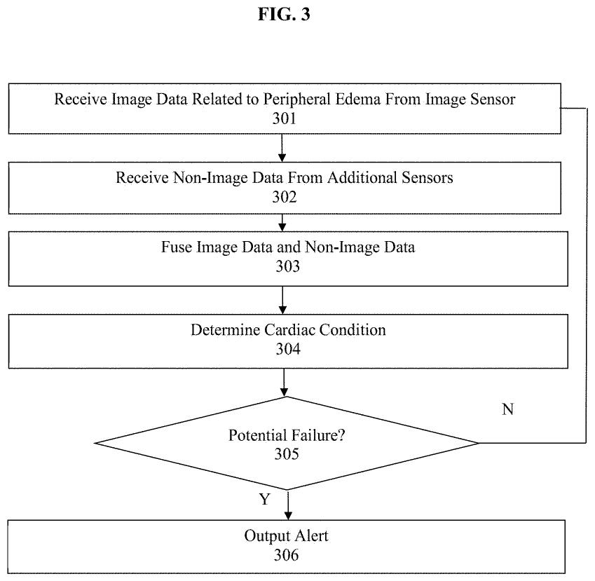

Accordingly, the memory of the disease monitoring computing device 101 can store application(s) that can include executable instructions that, when executed by the processor(s), cause the disease monitoring computing device 101 to perform actions, such as to perform the actions described and illustrated below with reference to FIG. 3. The application(s) can be implemented as modules or components of other application(s). Further, the application(s) can be implemented as operating system extensions, module, plugins, or the like.

Even further, the application(s) may be operative in a cloud-based computing environment. The application(s) can be executed within or as virtual machine(s) or virtual server(s) that may be managed in a cloud-based computing environment. Also, the application(s), and even the disease monitoring computing device itself, may be located in virtual server(s) running in a cloud-based computing environment rather than being tied to one or more specific physical network computing devices. Also, the application(s) may be running in one or more virtual machines (VMs) executing on the disease monitoring computing device 101. Additionally, in one or more embodiments of this technology, virtual machine(s) running on the disease monitoring computing device 101 may be managed or supervised by a hypervisor.

In this particular example, the memory 103 of the disease monitoring computing device 101 includes a fusion module 104, although the memory 103 can include other policies, modules, databases, or applications, for example. The fusion module 104 in this example is configured to fuse image data from the image sensor 106 with non-image data from one or more of the additional sensors, although the fusion module 104 could also fuse the image data with data received from one or more of the server devices 107. The fusion module 104 can fuse the image data from the image sensor 106 with data from any number and/or types of additional sensors 109. The fusion module 104 is configured to apply one or more data fusion algorithms to the image data from the image sensor 106 and the additional non-image data. By way of example only, the fusion module 104 may apply one or more of an image weighted Bayesian function, logistic regression, linear regression, regression with regularization, least angle regression, naïve Bayes, classification and regression tress (CART), support vector machines, relevance vector machines (RVM), neural network, or linear discriminant analysis to provide the fused data. The fusion module 104 advantageously fuses the image and non-image data to provide for disease monitoring as described in detail below with respect to the exemplary method illustrated in FIG. 3.

The communication interface 111 of the disease monitoring computing device operatively couples and communicates between the disease monitoring computing device, the image sensor, the additional sensors, the client devices and/or the server devices, which are all coupled together by the communication network(s) illustrated, although other types and/or numbers of communication networks or systems with other types and/or numbers of connections and/or configurations to other devices and/or elements can also be used.

By way of example only, the communication network(s) shown in FIG. 1 can include local area network(s) (LAN(s)) or wide area network(s) (WAN(s)), and can use TCP/IP over Ethernet and industry-standard protocols, although other types and/or numbers of protocols and/or communication networks can be used. The communication network(s) in this example can employ any suitable interface mechanisms and network communication technologies including, for example, teletraffic in any suitable form (e.g., voice, modem, and the like), Public Switched Telephone Network (PSTNs), Ethernet-based Packet Data Networks (PDNs), combinations thereof, and the like.

The disease monitoring computing device can be a standalone device or integrated with one or more other devices or apparatuses, such as the image sensor or the one or more of the server devices or the client devices, for example. In one particular example, the disease monitoring computing device can include or be hosted by one of the server devices or one of the client devices, and other arrangements are also possible.

In this example, the image sensor is a device configured for monitoring fluid, such as in the case of edema, in the tissue of a patient. By way of example, the image sensor may be the system for detecting edema as disclosed in U.S. Patent Application Publication No. 2019/0231260, the disclosure of which is hereby incorporated by reference in its entirety. The image sensor allows for obtaining image data that can be utilized to measure peripheral edema for monitoring disease activity, such as potential heart failure. The image sensor can advantageously be used to measure peripheral edema in non-shin locations, such as from the patient's forearm. Although the image sensor is illustrated as a standalone device, the image sensor could be incorporated in the disease monitoring computing device, or in one of the server devices or client devices.

In other embodiments, a spectral sensor is configured for monitoring edema in the tissue of a patient. The spectral sensor is not limited and includes sensors that determine the spectra that is emitted from tissue that of a patient that is suspected or is known to have edema. The edema is monitored by way of analysis of the spectra that is reflected from the tissue, and includes Raman spectroscopy or Fourier Transform Infrared Spectroscopy (FTIR), or any combination of these or similar techniques. In certain implementations of the spectral sensor, the spectral sensor generates spectral data, and the spectral data can be fused with additional data that is collected by additional sensors to thereby generate a fused data set.

The additional sensors are sensors known in the art that may be used to collect non-image data relevant to disease monitoring. By way of example only, the additional sensors can include a pulse oximeter (or other sensors configured to measure hemodynamics), an electrocardiogram (ECG or EKG), a sensor for measuring thoracic impedance, or an implantable disease monitoring device, although any other sensors capable of measuring data relevant to disease activity could be employed.

Each of the server devices in this example includes processor(s), a memory, and a communication interface, which are coupled together by a bus or other communication link, although other numbers and/or types of network devices could be used. The server devices in this example can host data associated with the additional sensors, or other patient data.

The client devices in this example include any type of computing device that can interface with the disease monitoring computing device to submit data and/or receive GUI(s). Each of the client devices in this example includes a processor, a memory, and a communication interface, which are coupled together by a bus or other communication link, although other numbers and/or types of network devices could be used.

The client devices can run interface applications, such as standard web browsers or standalone client applications, which may provide an interface to communicate with the disease monitoring computing device via the communication network(s). The client devices may further include a display device, such as a display screen or touchscreen, and/or an input device, such as a keyboard, for example. In one example, the client devices can be utilized by hospital staff to facilitate improved disease monitoring, although other types of client devices utilized by other types of users, such as the patient can also be used in other examples. In one example, the client devices receive data including patient information, such as name, date of birth, medical history, etc., for example. In other examples, this information is stored on one of the server devices.

In one embodiment, the disease monitoring computing device is configured to monitor for atrial fibrillation. When monitoring for atrial fibrillation, atrial fibrillation is designated as the hypothesis disease, and data is collected from one or more image sensors that show one or more of arterial stiffness, subcutaneous lipid build up, intravascular lipid build up, and the width of veins. Such imagery can be in the visible spectrum, which is referred to as RGB images or RBG video, or it can be hyperspectral imagery. For example, images or video in the visible spectrum can, when the image or video includes at least one reference, be processed by one or more processors to determine the width of an artery or vein. The artery that is measured by the RGB video or RGB image can be a visible artery or vein, such as one of the jugular veins. In certain embodiments, the visible artery or vein is shown in the same image or video as a known measurement reference, such as a scale or an object having a known dimension. When measuring atrial fibrillation, additional measurements can also include one or more of a blood pressure measurement, an atrial stiffness measurement, a blood composition test, echocardiography, electrophysiology study, sleep study, stress study, or transesophageal echocardiography (TEE) walking test. One or more of these tests can be fused with at least one image from an image sensor. When the measurements are gathered, one or more of the measurements are fused with an image or video and analyzed to determine whether the patient has the hyposthesis disease. Such fusion and measurement as described above can alternatively be fused in other ways for other purposes as will be apparent to those of ordinary skill in the art.

In some embodiments, the presence of anemia can be confirmed. Confirming the presence of anemia can include the use of hyperspectral images or video, but such images or video may also be fused with measurements of iron and/or oxygen in the blood. Furthermore, the imaging information can be fused with a depiction of one or more of blood oxygen saturation and/or iron concentrations.

In another embodiment, the presence of kidney disease can be confirmed. The confirmation of kidney disease can include hyperspectral images or video which are fused with other measurements, such as respiratory stress tests, blood pressure, and blood oxygen saturation. One or more of the preceding measurements may be fused with one or more of hyperspectral images or video.

In another embodiment, hydration is confirmed. While such monitoring may not identify a potential disease indication, it may be desirable to know a person's hydration level to assess sports performance or the person's general health and well being. To monitor hydration or to determine hydration, hyperspectral or RGB images or video are fused with at least one of a respiration parameter, blood pressure, VO₂ max (the maximum rate of oxygen consumption during exercise), or another indicator of respiratory health. Other tests can include one or more of weigh-ins (before and/or after physical activity), blood or plasma osmolality, bio-impedance, saliva tracking and thirst assessment, urine color, and urine specific gravity (USG), each of which can be fused to images or video in one or more of the hyperspectral region or the visible (RGB) region.

In another embodiment, a patient is monitored for sepsis. When monitoring for sepsis, sepsis is designated as the hypothesis disease, and data is collected from one or more image sensors that show abdominal fluid accumulation in the thoracic cavity. Such imaging information can also be fused with one or other measurements.

Although the exemplary environment with the disease monitoring computing device, server devices, client devices, image sensor, additional sensors, and communication network(s) are described and illustrated herein, other types and/or numbers of systems, devices, components, and/or elements in other topologies can be used. It is to be understood that the systems of the examples described herein are for exemplary purposes, as many variations of the specific hardware and software used to implement the examples are possible, as will be appreciated by those skilled in the relevant art(s).

One or more of the devices depicted in the environment, such as the disease monitoring computing device, client devices, or server devices, for example, may be configured to operate as virtual instances on the same physical machine. In other words, one or more of the disease monitoring computing device, client devices, or server devices may operate on the same physical device rather than as separate devices communicating through communication network(s). Additionally, there may be more or fewer disease monitoring computing devices, client devices, or server devices than illustrated in FIG. 1.

In addition, two or more computing systems or devices can be substituted for any one of the systems or devices in any example. Accordingly, principles and advantages of distributed processing, such as redundancy and replication also can be implemented, as desired, to increase the robustness and performance of the devices and systems of the examples. The examples may also be implemented on computer system(s) that extend across any suitable network using any suitable interface mechanisms and traffic technologies, including by way of example only wireless networks, cellular networks, PDNs, the Internet, intranets, and combinations thereof.

The examples may also be embodied as one or more non-transitory computer readable media (e.g., the memory) having instructions stored thereon for one or more aspects of the present technology as described and illustrated by way of the examples herein. The instructions in some examples include executable code that, when executed by one or more processors (e.g., the processor(s)), cause the processor(s) to carry out steps necessary to implement the methods of the examples of this technology that are described and illustrated herein.

An exemplary method of disease monitoring using fused sensor data, including both image data and non-image data, will now be described with reference to FIG. 3. First, the disease monitoring computing device receives image data from an image sensor configured for monitoring edema in a patient in block 301. By way of example, the image sensor may be the system for detecting edema as disclosed in U.S. Patent Application Publication No. 2019/0231260, the disclosure of which is hereby incorporated by reference in its entirety. For example, the image sensor can include a light source configured to irradiate a tissue of the patient with light. The tissue could be located in the patient's forearm, although other tissue locations could be utilized. The image sensor also includes a detector configured to collect reflected light from the tissue of the patient and generate the image data associated with the reflected light. The image data can be received and used to calculate intensity values to determine whether the tissue exhibits symptoms of edema as described in U.S. Patent Application Publication No. 2019/0231260, the disclosure of which is hereby incorporated by reference in its entirety. In one example, the image data can be processed on the disease monitoring computing device.

Next, in block 302 the disease monitoring computing device receives additional data from one or more additional sensors configured to monitor one or more factors related to disease activity of the patient. In one example, the data received from the additional sensors in non-image data relevant to the disease condition of the patient. The additional sensors can include can include a pulse oximeter (or other sensors configured to measure hemodynamics), an electrocardiogram (ECG or EKG), a sensor for measuring thoracic impedance, breathing rate monitor, patient weight monitor (such as a scale), or an implantable disease monitoring device, although any other sensors capable of measuring data relevant to disease activity could be employed. In another example, the disease monitoring computing device can receive additional data from one or more of the server devices or client devices.

In block 303, the disease monitoring computing device fuses the received image data from the image sensor with the received additional data from the one or more additional sensors to generate a fused data set. The disease monitoring computing device utilizes applies one or more data fusion algorithms the received image data and the received additional data in order to generate the fused data set. By way of example, the disease monitoring computing device uses one or more of an image weighted Bayesian function, logistic regression, linear regression, regression with regularization, naïve Bayes, classification and regression tress, support vector machines, relevance vector machines, (RVM), least angle regression, linear discriminant analysis, or a neural network, although other data fusion techniques or systems may be employed.

Next, in block 304 the disease monitoring computing device determines a disease condition for the patient based on the fused data set. In one example, the disease monitoring computing device can apply one or more machine learning models to the fused data set to determine the disease condition. The disease monitoring computing device can store and/or obtain training data related to the disease condition determination based on fused data sets from image and non-image sensors. The disease monitoring computing device can generate or train a machine learning model based on the training data and the generated fused data set. In one example, the machine learning model is a neural network, such as an artificial or convolutional neural network, although other types of neural networks or machine learning models can also be used in other examples. In one example, the neural network is a fully convolutional neural network. The generated fused data set in the previous step can be evaluated using the trained model to determine the patient's disease condition.

Next, in one implementation depicted by block 305, the disease monitoring computing device identifies whether there is a potential failure. If no, the process is repeated from the beginning to collect additional data for monitoring the disease condition of the patent. The generated fused data set can then be used to further train the machine learning model. If a potential failure is identified, then an alert is output to one or more of the server devices and/or client devices. Although an example of provide information based on a potential failure is described, the determined disease condition can also be utilized to provide treatment regimen changes, or other information to the patient and/or health care provided.

With this technology, image data related to peripheral edema is fused with non-image sensor data relevant to disease monitoring to provide a practical application of a more efficient and effective method of monitoring disease activity. The technology advantageously combines the multiple sensor logics to provide earlier warning of potential disease failure, or other disease issues. The technology can further be used to provide improved tele-monitoring of patients with heart-related issues.

In certain embodiments of the disclosure, the disease monitoring computing device generates an objective measure of disease health in block 306. For example, the objective measure can be a relative probability of a disease condition. In other examples, the objective measure is a composite indicator that indicates the degree of severity of a patient's edema. By generating an objective, numerical measure of a patient's disease health, including the severity of the patients edema, clinicians can better anticipate or adjust the level and types of intervention that are given to the patient. Still further, the disease monitoring computing device can record the generated objective measures over time, which enables clinicians to even further predict the health of a particular patient.

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. It will be further understood by those within the art that if a specific number of an introduced claim recitation is intended, such an intent will be explicitly recited in the claim, and in the absence of such recitation no such intent is present.

For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1. A method of disease monitoring, the method comprising:
receiving, by a disease monitoring computing device, image data from an image sensor configured for monitoring a hypothesis disease in a patient;
receiving, by the disease monitoring computing device, additional data from one or more additional sensors configured to monitor one or more factors related to disease activity of the patient;
fusing, by the disease monitoring computing device, the received image data with the received additional data from the one or more additional sensors to generate a fused data set; and
determining, by the disease monitoring computing device, a hypothesis disease for the patient based on the fused data set.

2. The method of claim 1, wherein fusing the received image data with the received additional data further comprises:
applying, by the disease monitoring computing device, one or more data fusion algorithms to the received image data and the received additional data.

3. The method of claim 2, wherein applying the data fusion algorithm comprises utilizing one or more of an image weighted Bayesian function, logistic regression, linear regression, regression with regularization, naïve Bayes, classification and regression tress, support vector machines, or a neural network.

4. The method of claim 1, wherein the received additional data is non-image data.

5. The method of claim 1, wherein the one or more additional sensors comprise one or more of a pulse oximeter, an electrocardiogram machine, a sensor for thoracic impedance, or an implantable disease monitoring device.

6. The method of claim 1, wherein determining the hypothesis disease further comprises:
identifying, by the disease monitoring computing device, one or more indications of a potential heart failure.

7. The method of claim 1, wherein the image sensor comprises:
a light source configured to irradiate a tissue of the patient with light; and
a detector configured to collect reflected light from the tissue of the patient and generate the image data associated with the reflected light;
wherein the method further comprises:
receiving, by the disease monitoring computing device, the image data associated with the reflected light;
calculating, by the disease monitoring computing device, intensity values for reflected light; and
determining, by the disease monitoring computing device, whether the tissue exhibits symptoms of edema.

8. The method of claim 7, wherein the tissue of the patient is located on a forearm of the patient.

9. The method of claim 1, wherein the image data is spectral data and the image sensor is a spectral sensor.

10. The method of claim 1, wherein the hypothesis disease includes at least one of lymphatic disease, kidney disease, peripheral vascular disease, protein deficiency (including protein S deficiency), chronic obstructive pulmonary disease, diabetes, sepsis, cancer such as breast cancer with lymph node metastasis, or stroke (including ischemic stroke, hemorrhagic stroke, or transient ischemic attack).

11. A non-transitory computer readable medium having stored thereon instructions for improved disease monitoring comprising executable code that, when executed by one or more processors, causes the one or more processors to:
receive image data from an image sensor configured for monitoring edema in a patient;
receive additional data from one or more additional sensors configured to monitor one or more factors related to disease activity of the patient;
fuse the received image data with the received additional data from the one or more additional sensors to generate a fused data set; and
determine a disease condition for the patient based on the fused data set.

12. The non-transitory computer readable medium of claim 11, wherein the processors fuse the received image data with the received additional data by applying one or more data fusion algorithms to the received image data and the received additional data.

13. The non-transitory computer readable medium of claim 12, wherein the data fusion algorithm includes one or more of an image weighted Bayesian function, logistic regression, linear regression, regression with regularization, naïve Bayes, classification and regression tress, support vector machines, or a neural network.

14. The non-transitory computer readable medium of claim 11, wherein the additional data that the processors receive is non-image data.

15. The non-transitory computer readable medium of claim 11, wherein the one or more additional sensors comprise one or more of a pulse oximeter, an electrocardiogram machine, a sensor for thoracic impedance, or an implantable disease monitoring device.

16. The non-transitory computer readable medium of claim 11, wherein determination of the disease condition further comprises:
identifying one or more indications of lymphatic disease, kidney disease, peripheral vascular disease, protein deficiency (including protein S deficiency), chronic obstructive pulmonary disease, diabetes, sepsis, cancer such as breast cancer with lymph node metastasis, or stroke (including ischemic stroke, hemorrhagic stroke, or transient ischemic attack).

17. A disease monitoring computing device comprising memory comprising programmed instructions stored thereon for disease monitoring and one or more processors coupled to the memory and configured to execute the stored programmed instructions, which when the programmed instructions are executed the disease monitoring computing device:
receives image data from an image sensor configured for monitoring edema in a patient;
receives additional data from one or more additional sensors configured to monitor one or more factors related to disease activity of the patient;
fuses the received image data with the received additional data from the one or more additional sensors to generate a fused data set; and
determines a disease condition for the patient based on the fused data set.

18. The disease monitoring computing device of claim 17, wherein the processors fuse the received image data with the received additional data by applying one or more data fusion algorithms to the received image data and the received additional data.

19. The disease monitoring computing device of claim 18, wherein the data fusion algorithm includes one or more of an image weighted Bayesian function, logistic regression, linear regression, regression with regularization, naïve Bayes, classification and regression tress, support vector machines, or a neural network.

20. The disease monitoring computing device of claim 17, wherein the additional data that the processors receive is non-image data.

21. The disease monitoring computing device of claim 17, wherein the one or more additional sensors comprise one or more of a pulse oximeter, an electrocardiogram machine, a sensor for thoracic impedance, an implantable disease monitoring device, or a breathing rate device.

22. The disease monitoring computing device of claim 17, wherein determining the disease condition further comprises:
identifying one or more indications including lymphatic disease, kidney disease, peripheral vascular disease, protein deficiency (including protein S deficiency), chronic obstructive pulmonary disease, diabetes, sepsis, cancer such as breast cancer with lymph node metastasis, or stroke (including ischemic stroke, hemorrhagic stroke, or transient ischemic attack).

23. The disease monitoring computing device of claim 17, wherein the image sensor comprises:
a light source configured to irradiate a tissue of the patient with light; and
a detector configured to collect reflected light from the tissue of the patient and generate the image data associated with the reflected light;
wherein the disease monitoring computing device further:
receives the image data associated with the reflected light;
calculates intensity values for reflected light; and
determines whether the tissue exhibits symptoms of edema.

24. The disease monitoring computing device of claim 17, wherein the image data is spectral data and the image sensor is a spectral sensor.
